(19) United States
(12) Patent Application Publication
(10) Pub. No.: US 2009/0285887 A1
(43) Pub. Date: Nov. 19, 2009

(54) Pharmaceutical Composition Comprising A Plurality of Mini-Tablets Comprising A Factor XA Inhibitor

(76) Inventors: Omar Abdelfattah Abu-Baker, King of Prussia, PA (US); Yong Hu, King of Prussia, PA (US); Kimberly Anne Lamey, King of Prussia, PA (US); Robert Francis Leposki, King of Prussia, PA (US); Rennan Pan, King of Prussia, PA (US); Kamlesh Rameshchandra Patel, King of Prussia, PA (US); Rahul Parashar Shukla, King of Prussia, PA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/440,740

(22) PCT Filed: Sep. 10, 2007
(86) PCT No.: PCT/EP07/59443

§ 371 Date: Mar. 11, 2009

Related U.S. Application Data
(60) Provisional application No. 60/825,314, filed on Sep. 12, 2006.

(51) Int. Cl. A61K 9/26; A61K 31/5377; A61P 43/00

(52) U.S. Cl. 424/469; 514/235.5

(57) ABSTRACT

A modified release pharmaceutical composition for oral administration comprising plural mini-tablets, comprising a therapeutically effective amount of a Factor Xa inhibitor within a matrix of polymer(s). The mini-tablets are suitably encapsulated within a gelatin capsule. A manufacturing process and method of use are also described.

The present invention relates to pharmaceutical compositions comprising an effective amount of a Factor Xa inhibitor, for example (E)-2-(5-chlorothien-2-yl)-N-{(3S)-1-[(1S)-1-methyl-2-morpholin-4-yl-2-oxoethyl]-2-oxopyrrolidin-3-yl}ethenesulfonamide (“Compound A”) or (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide (“Compound B”), and to their use in treating or preventing conditions for which a Factor Xa inhibitor is indicated.

### BACKGROUND OF THE INVENTION

Factor Xa is a member of the trypsin-like serine protease class of enzymes. It is a key enzyme in the coagulation cascade. A one-to-one binding of Factors Xa and Va with calcium ions and phospholipid converts prothrombin into thrombin. Thrombin plays a central role in the mechanism of blood coagulation by converting the soluble plasma protein, fibrinogen, into insoluble fibrin. The insoluble fibrin matrix is required for the stabilisation of the primary hemostatic plug. Many significant disease states are related to abnormal hemostasis. With respect to the coronary arterial vasculature, abnormal thrombus formation due to the rupture of an established atherosclerotic plaque is the major cause of acute myocardial infarction and unstable angina. Both treatment of an occlusive coronary thrombus by thrombolytic therapy and percutaneous transluminal coronary angioplasty (PTCA) are often accompanied by an acute thrombotic reclosure of the affected vessel which requires immediate resolution. With respect to the venous vasculature, a high percentage of patients undergoing major surgery in the lower extremities or the abdominal area suffer from thrombus formation in the venous vasculature which can result in reduced blood flow to the affected extremity and a pre-disposition to pulmonary embolism. Disseminated intravascular coagulopathy commonly occurs within both vascular systems during septic shock, certain viral infections and cancer and is characterised by the rapid consumption of coagulation factors and systemic coagulation which results in the formation of life-threatening thrombi occurring throughout the vasculature leading to widespread organ failure. Beyond its direct role in the formation of fibrin rich blood clots, thrombin has been reported to have profound bioregulatory effects on a number of cellular components within the vasculature and blood, (Shuman, M. A., Ann. NY Acad. Sci., 405: 349 (1986)).

A Factor Xa inhibitor may be useful in the treatment of acute vascular diseases (Turpie (2007) Arterioscler. Throm. Vasc. Biol. 27:1238-47; Eriksson et al. (2006) Drugs 66(11):1411-1429) such as acute coronary syndromes (for example primary and secondary prevention of myocardial infarction and unstable angina and treatment of prothrombotic sequalae associated with myocardial infarction or heart failure), thromboembolism including venous thromboembolism (VTE) (deep vein thrombosis (DVT) and pulmonary embolism (PE)), acute vessel closure associated with thrombolytic therapy and percutaneous transluminal coronary angioplasty, transient ischemic attacks, peripheral arterial occlusion, prevention of vessel luminal narrowing (restenosis), and the prevention of thromboembolic events associated with atrial fibrillation, e.g. stroke (stroke prevention in patents with atrial fibrillation, SPAF). Factor Xa inhibitors may also be useful in preventing thrombosis and complications in patients genetically predisposed to arterial thrombosis or venous thrombosis and patients that have a disease-associated predisposition to thrombosis (e.g. type 2 diabetics). Thrombin has been reported to contribute to lung fibroblast proliferation, thus, Factor Xa inhibitors could be useful for the treatment of some pulmonary fibrotic diseases. Factor Xa inhibitors could also be useful in the treatment of tumour metastasis, by suppressing coagulation and thus preventing fibrin deposition and its concommittant facilitation of metastasis. A Factor Xa inhibitor may also have utility as an anti-inflammatory agent through its inhibition of FXa mediated activation of protease-activated receptors (PAR 1-4). A Factor Xa inhibitor may also have utility as an anti-atherosclerotic agent through the suppression of platelet-activation. Thrombin can induce neurite retraction and thus Factor Xa inhibitors may have potential in neurogenerative diseases such as Parkinson's and Alzheimer's disease (Haas et al. (1997) Biochim. Biophys. Acta. 1343(1): 85-94). Factor Xa inhibitors may also have utility as anticoagulant agents in connection with the preparation, storage, fractionation or use of whole blood. They have also been reported for use in conjunction with thrombolytic agents, thus permitting the use of a lower dose of thrombolytic agent.

Factor Xa inhibitors include those disclosed in PCT publications WO02100886, WO02100830, WO03043981, WO03053925, WO04052851, WO04052878 WO2004110997, WO2004110434, WO2004111045, WO2004110435, WO2006027186, WO2006108709 and WO2007059952 incorporated herein by reference. Factor Xa inhibitors are also discussed in the following publications: Watson et al. (2006) Bioorg. Med. Chem. Lett. 16(14):3784-8; Young et al. (2006) Bioorg. Med. Chem. Lett. 16(23) 5953-7; Senger et al. (2006) Bioorg. Med. Chem. Lett. 16(22): 5731-5; Chan et al. (2007) J. Med. Chem. 50(7): 1546-57; Young et al. (2007) Bioorg. Med. Chem. Lett. 17(10):2927-30; and Senger et al. (2007) 17(10):2931-4. For example, (E)-2-(5-Chlorothien-2-yl)-N-{(3S)-1-[(1S)-1-methyl-2-morpholin-4-yl-2-oxoethyl]-2-oxopyrrolidin-3-yl}ethenesulfonamide and/or a pharmaceutically acceptable solvate thereof, is a FXa inhibitor disclosed in WO02/100886 and WO02/100830. (E)-2-(5-Chlorothien-2-yl)-N-{(3S)-1-[(1S)-1-methyl-2-morpholin-4-yl-2-oxoethyl]-2-oxopyrrolidin-3-yl}ethenesulfonamide has the structure shown below (Compound A, Formula I):

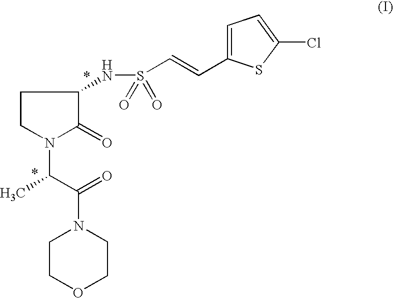

(E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide and/or a pharmaceutically acceptable solvate thereof, is a FXa inhibitor disclosed in WO2007059952 and has the structure shown below (Compound B, Formula II):

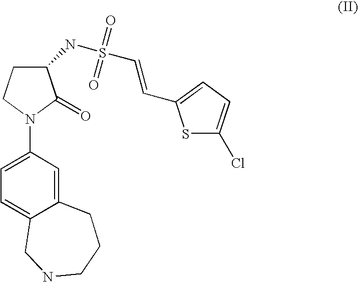

There is a need for modified release compositions of Factor Xa inhibitors having particular release profiles. The effect of food on the absorption profile of the Factor Xa inhibitor should also be minimised. The present invention provides a pharmaceutical composition for Factor Xa inhibitors, which alleviates food effect and is capable of providing therapeutically effective levels of a Factor Xa inhibitor over extended periods of time after oral administration, e.g. for at least 12 or 24 hours, thus enabling twice daily dosing or once daily dosing.

Schmitz et al. (2005) Journal of Pharmaceutical Sciences, 94(5), 966-973 describe mini-tablet formulations based on thiolated polycarbophil and hydroxyethylcellulose (HEC) polymers and having a diameter of 2 mm and a thickness of 1 mm to provide a stomach targeted oral delivery system for low molecular weight heparin (LMWH), a hydrophilic macromolecular polysaccharide which has Factor Xa inhibitory activity. Similarly, WO0/48589 (Emisphere) describes a solid oral dosage form containing a heparin drug in admixture with a carrier such that the dosage form protects the carrier from precipitation during transit through the low PH regions of the GI track, thus enabling concurrent presentation of the heparin drug and the carrier in the GI track to facilitate the absorption and/or enhance the bioavailability of the heparin drug. The solid dosage forms described therein include tablets and multiparticulates, e.g. mini-tablets. Other publications describing Factor Xa inhibitors mention microtablets or mini-tablets as possible dosage forms (for example U.S. Pat. No. 6,794,412B1 and WO2006/100565) but do not describe the pharmaceutical formulations of the present invention which alleviate food effect and are capable of providing therapeutically effective levels of a Factor Xa inhibitor over extended periods of time after oral administration.

### SUMMARY OF THE INVENTION

The present invention provides modified release pharmaceutical compositions for oral administration comprising a plurality of mini-tablets (also known as “mini-tabs”), said mini-tablets having a diameter of less than 5 mm and comprising a therapeutically effective amount of a Factor Xa inhibitor, e.g. Compound A, Compound B, within a matrix of polymer(s).

The present invention also provides modified release pharmaceutical compositions for oral administration comprising a Factor Xa inhibitor and characterized by one or both of the following properties:

a) an in vivo maximum plasma concentration (Cₘₐₓ) following single oral dose administration to healthy adult humans wherein a ratio of Cₘₐₓ Geometric Mean Ratio (GMR) Fasted:Fed is between 0.90 to 1.10; and
b) an in vivo area under the curve (AUC) following single oral dose administration to healthy adult humans wherein a ratio of AUC GMR Fasted:Fed is between 0.90 to 1.10.

In one embodiment, the modified release pharmaceutical composition comprises a plurality of enteric coated mini-tablets. The enteric coating may comprise a methacrylic acid copolymer, for example Eudragit (e.g. Eudragit L30D55). The mini-tablet may further comprise a matrix polymer and may suitably further comprise a filler, a lubricant, and a glidant (one more such components may be utilized). For example, the composition may comprise from 5-50% of a Factor Xa inhibitor, from 20-50% matrix polymer, from 20-50% filler, from 0.1-5% lubricant, and from 0.1-5% glidant, based on total weight of the composition. Suitably, the matrix polymer is hypromellose (also known as hydroxypropyl methylcellulose or “HPMC”), the filler is microcrystalline cellulose, the lubricant is magnesium stearate, and the glidant is colloidal silicon dioxide.

The present invention also provides a pharmaceutical composition of the invention for the manufacture of a medicament for the treatment of a patient suffering from a condition susceptible to amelioration by a Factor Xa inhibitor, a pharmaceutical composition of the invention for use in the treatment of a condition susceptible to amelioration by a Factor Xa inhibitor and a method of treating a patient suffering from a condition susceptible to amelioration by a Factor Xa inhibitor comprising administering a pharmaceutical composition of the invention.

### BRIEF DESCRIPTION OF THE FIGURES

FIG. 1 shows a diagram of an enteric coated mini-tablet pharmaceutical composition according to the present invention. Referring to FIG. 1, an encapsulated composition 10 overall comprises a gelatine capsule 11. Within capsule 11 are plural mini-tabs 12 to be further described below. These mini-tabs 12 have a diameter (as defined above) of 3.2 mm (round standard convex) and are enteric coated to dissolve at pH>5.5 i.e. after they have left the stomach. The capsule 11 may be filled with an overfill 13 of microcrystalline cellulose.

FIG. 2 is a graph comparing dissolution profiles of monolithic modified release dosage forms with and without microcrystalline cellulose.

FIG. 3 is a graph from a human PK study showing time course of median plasma concentration following oral administration of 150 mg of Compound A administered as an enteric coated mini-tablet pharmaceutical composition under a fasted state. Each line of data points represents an individual subject's PK data.

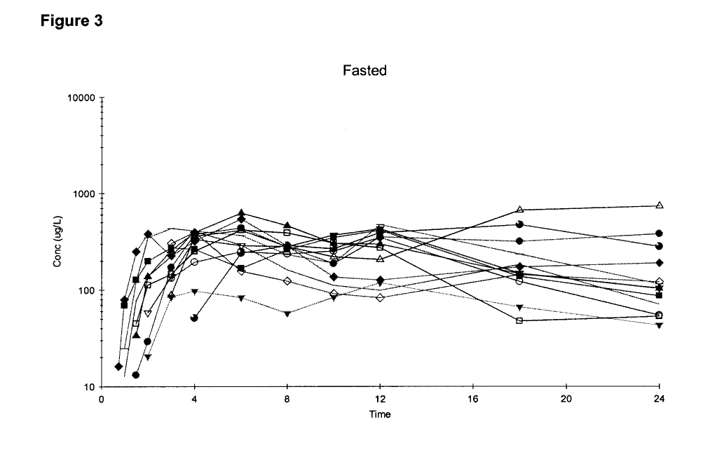

FIG. 4 is a graph from a human PK study showing time course of median plasma concentration following oral administration of 150 mg of Compound A administered as an enteric coated mini-tablet pharmaceutical composition with a standard meal. Each line of data points represents an individual subject's PK data.

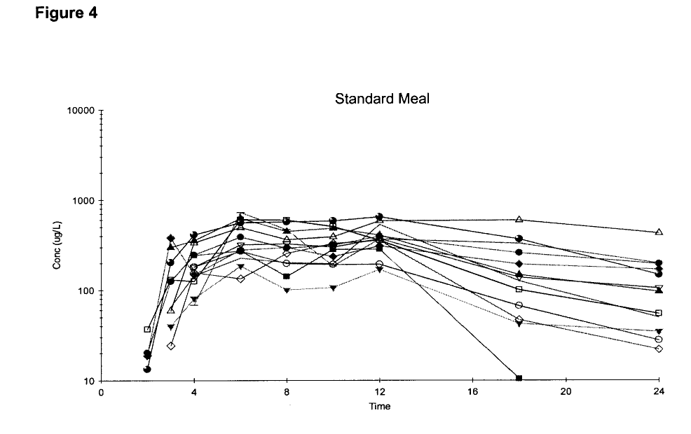

FIG. 5 is a graph from a human PK study showing time course of median plasma concentration following oral administration of 150 mg of Compound A administered as an enteric coated mini-tablet pharmaceutical composition with a high fat meal. Each line of data points represents an individual subject's PK data.

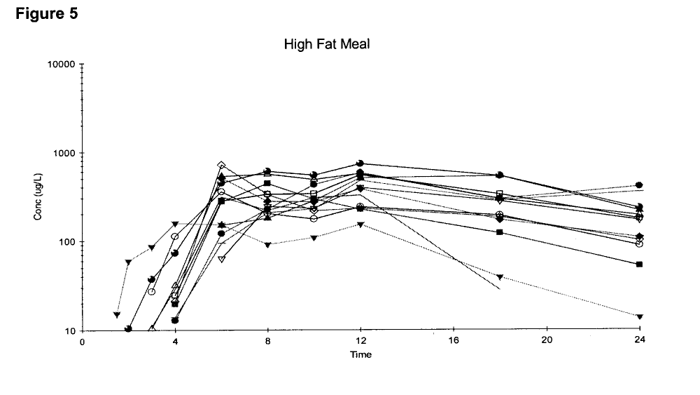

### DETAILED DESCRIPTION OF THE INVENTION

The present invention relates to Factor Xa inhibitors, for example, Factor Xa inhibitors disclosed in PCT publications WO02100886, WO02100830, WO03043981, WO03053925, WO04052851, WO04052878 WO2004110997, WO2004110434, WO2004111045 and WO2004110435 such as (E)-2-(5-Chlorothien-2-yl)-N-{(3S)-1-[(1S)-1-methyl-2-morpholin-4-yl-2-oxoethyl]-2-oxopyrrolidin-3-yl}ethenesulfonamide and/or a pharmaceutically acceptable solvate thereof (Compound A) and (E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide (Compound B). It will be understood that Compound A includes solvates (including hydrates) of Compound A, crystalline and non-crystalline forms; Compound B includes solvates (including hydrates) of Compound B, crystalline and non-crystalline forms. The individual stereoisomers (enantiomers and diastereoisomers) and mixtures of these are within the scope of the present invention.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of Compound A and Compound B are within the scope of the invention.

Other Factor Xa inhibitors within the scope of the present invention include: 4,5,6,7-tetrahydro-1-(4-methoxyphenyl)-7-oxo-6-[4-(2-oxo-1-piperidinyl)phenyl]-1H-pyrazolo[3,4-c]pyridine-3-carboxamide (Apixaban) 5-chloro-N-({(5S)-2-oxo-3-[4-(3-oxo-4-morpholinyl)phenyl]-1,3-oxazolidin-5-yl}-methyl)-2-thiophenecarboxamide (Rivaroxaban); (2S)-2-(4-{[(3S)-1-(aminocarbonyl)-3-pyrrolidinyl]oxy}phenyl)-3-{7-[amino(imino)methyl]-2-naphthalenyl}propanoic acid (DX-9065a); N-(2-({5-[amino(imino)methyl]-2-hydroxyphenyl}oxy)-3,5-difluoro-6-{[3-(1-methyl-4,5-dihydro-1H-imidazol-2-yl)phenyl]oxy}-4-pyridinyl)-N-methylglycine (ZK807834, Fidexaban); 1-[3-(aminomethyl)phenyl]-N-[3-fluoro-2′-(methylsulfonyl)-4-biphenylyl]-3-(trifluoromethyl)-1H-pyrazole-5-carboxamide (DPC-423); 1-[2-(aminomethyl)phenyl]-N-[3-fluoro-2′-(methylsulfonyl)-4-biphenyl]-3-(trifluoromethyl)-1H-pyrazole-5-carboxamide (DPC-602); 1-(3-amino-1,2-benzisoxazol-5-yl)-N-[4-[2-[dimethylamino]methyl]-1H-imidazol-1-yl]-2-fluorophenyl-3-(trifluoromethyl)-1H-pyrazol-5-carboxamide (razaxaban); N-[2′-(aminosulfonyl)-3-fluoro-4-biphenylyl]-1-(2,7a-dihydro-1,2-benzisoxazol-5-yl)-1H-tetrazole-5-carboxamide (SR374); 4-{[(E)-2-(5-chloro-2-thienyl)ethenyl]sulfonyl}-1-(1H-pyrrolo[3,2-c]pyridin-2-ylmethyl)-2-piperazinone (RPR209685); (2E)-3-(1-amino-7-isoquinolinyl)-N-[2′-(aminosulfonyl)-3-bromo-4-biphenylyl]-2-fluoro-2-butenamide; (2E)-N-[2′-(aminosulfonyl)-3-bromo-4-biphenylyl]-2-fluoro-3-{3-[(Z)-(hydroxyamino)(imino)methyl]phenyl}-2-butenamide; N-[2′-(aminosulfonyl)-4-biphenylyl]-2-[1-(3-fluoro-2-naphthalenyl)-3-methyl-1H-pyrazol-5-yl]acetamide; 3-methyl-N-[2′-(methylsulfonyl)-4-biphenylyl]-1-[3-(methylsulfonyl)-2-naphthalenyl]-1H-pyrazole-5-carboxamide; [(({7-[amino(imino)methyl]-2-naphthalenyl}methyl){4-[(1-ethanimidoyl-4-piperidinyl)oxy]phenyl}amino)sulfonyl]acetic acid (YM60828); N-({7-[amino(imino)methyl]-2-naphthalenyl}methyl)-N-{4-[(1-ethanimidoyl-4-piperidinyl)oxy]phenyl}-b-alanine (YM169964); N-{3-[amino(imino)methyl]phenyl}-2-{6-[(1-ethanimidoyl-4-piperidinyl)oxy]-2,2-dioxido-4-oxo-3,4-dihydro-1H-2,1,3-benzothiadiazin-1-yl}acetamide (YM169920); 2-(R)-(3-Carbamimidoylbenzyl)-3-(R)-[4-(1-oxypyridin-4-yl)benzoylamino]-butyric acid methyl ester (Otamixaban); 1-amino-N-{2-oxo-1-phenyl-2-[4-(4-pyridinyl)-1-piperazinyl]ethyl}-7-isoquinolinecarboxamide (PMD3112); and N-{(1R)-2-[4-(1-methyl-4-piperidinyl)-1-piperazinyl]-2-oxo-1-phenylethyl}-1H-indole-6-carboxamide (LY517717).

In one aspect of the invention, the Factor Xa inhibitor is other than a heparin or heparinoid drug (such as low molecular weight heparin, LMWH). In another aspect of the invention, the Factor Xa inhibitor is a small molecule Factor Xa inhibitor, i.e. not a polysaccharide or polypeptide.

As used herein, the term “pharmaceutically acceptable” means a compound or composition which is suitable for pharmaceutical use.

As used herein, “modified release composition” means a dosage form in which the release of a Factor Xa inhibitor is modified (or controlled) over a period of time compared to an immediate release formulation. Modified, can mean, for example, that the release of a Factor Xa inhibitor is extended for longer than it would be in an immediate release composition. For example, a modified release composition may provide that blood (e.g. plasma) levels of a Factor Xa inhibitor are maintained within a therapeutic range but below toxic levels for at least 12 hours, suitably at least 24 hours. For example, if a modified release composition possesses release properties and sufficient drug to maintain a drug concentration for twelve or more hours, that would desirably enable dosing twice daily, or less frequently each day.

As used herein, the term “diameter” means the greatest longitudinal dimension.

As used herein, the term “dissolution profile” means a plot of the cumulative amount of a Factor Xa inhibitor released as a function of time. The dissolution profile can, for example, be measured utilizing the Drug Release Test which incorporates standard test conditions according to USP or Ph Eur specifications, specifically according to USP <711> using Apparatus I, II or III.

As used herein, the term “fasted” means an overnight fast of at least 10 hours prior to drug administration with 240 mL (8 fluid ounces) of water and no food allowed for at least 4 hours post-dose. Water is permitted as desired, except for one hour before and after drug administration.

As used herein, the term “fed” means either a standard meal or high fat meal has been administered after an overnight fast of at least 10 hours and a meal starting 30 minutes prior to drug administration. The meal should be consumed in less than 30 minutes and drug administered 30 minutes after the start of the meal. No food is permitted for at least 4 hours post-dose. Water is permitted as desired, except for one hour before and after drug administration.

As used herein, the term “standard meal” means a light breakfast of approximately 321 calories and in compliance with FDA Guidance for Industry: Food-Effect Bioavailability and Fed Bioequivalence Studies.

As used herein, the term “high fat meal” means a high fat breakfast of approximately 682 calories and in compliance with FDA Guidance for Industry: Food-Effect Bioavailability and Fed Bioequivalence Studies.

As used herein, the term “matrix” means a composition in which the drug is embedded or dispersed in water soluble or insoluble polymers in order to achieve extended release of the drug. The mechanisms of the drug release generally involve drug diffusion through viscous gel layer, or tortuous channels; and/or drug dissolution via gradual system erosion or degradation. Suitably, the matrix comprises swellable/erodable polymers, for example hydrophilic polymers which in contact with the water form a gel of high viscosity.

As used herein, the term “enteric coating” means a coating which delays the release of the active agent from the mini-tablet until it reaches the intestine and releases drug in the duodenum, ileum and/or cecum/colon. Although most enteric coatings are generally known in the art to be pH-sensitive coatings, as used herein the term “enteric coating” includes both coatings that are pH-sensitive and coatings that are pH-independent. More particularly, the term “enteric coating” as used herein indicates that the coating is one that is selected for its ability to deliver active ingredients to the post-stomach gastrointestinal (GI) tract.

Release forms may also be characterized by their pharmacokinetic parameters. As used herein, the term “pharmacokinetic parameters” describes the in vivo characteristics of a Factor Xa inhibitor over time, including for example, the in vivo dissolution characteristics and plasma concentration of a Factor Xa inhibitor. By “Cₘₐₓ” is meant the measured concentration of a Factor Xa inhibitor in the plasma at the point of maximum concentration. By “C₁₂” is meant the concentration of the active agent in the plasma at 12 hours. By “C₂₄” is meant the concentration of the active agent in the plasma at 24 hours. The term “Tₘₐₓ” refers to the time at which the concentration of a Factor Xa inhibitor in the plasma is the highest. “AUC” is the area under the curve of a graph of the concentration of a Factor Xa inhibitor (typically plasma concentration) vs. time, measured from one time to another.

In one embodiment, the pharmaceutical composition of the present invention provides an in vivo maximum plasma concentration (Cₘₐₓ) following single oral dose administration to healthy adult humans wherein a ratio of Cₘₐₓ GMR Fasted:Fed is 0.90 to 1.15, (for example 0.90 to 1.10, 0.95 to 1.15, 0.95 to 1.10, 1.00 to 1.15, or 1.00 to 1.10).

In one embodiment, the pharmaceutical composition of the present invention provides an in vivo area under the curve (AUC) following single oral dose administration to healthy adult humans wherein a ratio of AUC GMR Fasted:Fed is 0.90 to 1.15 (for example 0.90 to 1.10, 0.90 to 1.05, 0.95 to 1.15, 0.95 to 1.10 or 0.95-1.05).

In one aspect of the invention, the present invention provides a pharmaceutical composition for oral administration comprising a Factor Xa inhibitor and characterized by the following properties:

a) an in vivo maximum plasma concentration (Cₘₐₓ) following single oral dose administration to healthy adult humans wherein a ratio of Cₘₐₓ Geometric Mean Ratio (GMR) Fasted:Fed is between 0.90 to 1.15 (for example 0.90 to 1.10, 0.95 to 1.15, 0.95 to 1.10, 1.00 to 1.15 or 1.00 to 1.10); and
b) an in vivo area under the curve (AUC) following single oral dose administration to healthy adult humans wherein a ratio of AUC GMR Fasted:Fed is between 0.90 to 1.15 (for example 0.90 to 1.10, 0.90 to 1.05, 0.95 to 1.15, 0.95 to 1.10 or 0.95-1.05).

In another aspect, the present invention provides pharmaceutical compositions comprising a Factor Xa inhibitor as described above and further characterized by having a dissolution profile wherein at 6 hours after combining the modified release composition with a dissolution medium under standard test conditions less than 50%, suitably less than 40%, or 30% of a Factor Xa inhibitor is released (e.g. 5 to 50%, 5 to 40%, 5 to 30%, 5 to 20%, 10 to 50%, 10 to 40%, 10 to 30%, 10 to 20% or 20 to 40%). In another embodiment, the modified release composition of the invention has a dissolution profile such that at 6 hours after combining the modified release composition with a dissolution medium under standard test conditions more than 50%, suitably more than 60%, or 70% of the pharmaceutical composition is remaining (e.g. 50 to 95%, 60 to 95%, 70 to 95%, 80 to 95%, 50 to 90%, 60 to 90%, 70 to 90% or 80 to 90%). In another embodiment, the modified release composition of the invention has a dissolution profile such that at 12 hours after combining the modified release composition with a dissolution medium under standard test conditions less than 80%, suitably less than 70%, 60%, 50%, or 40% of the Factor Xa inhibitor is released (e.g. 30 to 80%, 30 to 70%, 30 to 60%, 30 to 50% or 30 to 40%). In another embodiment, the modified release composition of the invention has a dissolution profile such that at 24 hours after combining the modified release composition with a dissolution medium under standard test conditions more than 30%, suitably more than 40%, or 50% of the pharmaceutical composition is remaining (e.g. 30 to 75%, 40 to 75%, 50 to 75%, 60 to 75%, 30 to 70%, 40 to 70%, 50 to 70% or 60 to 70%).

In another aspect, the present invention provides modified release compositions comprising a Factor Xa inhibitor as described above and further characterized by having a maximum plasma concentration (Cₘₐₓ) and a plasma concentration at 24 hours after administration or a single oral dose to healthy adult humans, wherein a ratio of Cₘₐₓ to C₂₄ is less than 20:1 (for example less than 15:1, or less than 5:1). In another embodiment the modified release composition of the present invention provides an in vivo maximum plasma concentration (Cₘₐₓ) following single oral dose administration (150 mg) to healthy adult humans that is less than 900 ng/mL (e.g. less than 800 ng/mL, or less than 740 ng/mL). In another embodiment, the modified release composition of the invention provides an in vivo plasma concentration following single oral dose (150 mg) administration to healthy adult humans at C₂₄ of at least 30 ng/mL (e.g. at least 40 ng/mL, at least 45 ng/mL or at least 100 ng/mL).

In one aspect of the invention, the pharmaceutical composition enables the Factor Xa inhibitor to be absorbed throughout the GI tract, i.e. in the duodenum (proximal small intestine), ileum (distal small intestine) and cecum/colon. Preliminary pharmacokinetic analysis has demonstrated that Compound A may be absorbed throughout the GI tract. Accordingly, this comprises a further aspect of the invention.

The pharmaceutical compositions of the present invention suitably provide therapeutically effective levels of a Factor Xa inhibitor over extended periods of time after oral administration, e.g. for at least 12 or 24 hours, thus enabling twice daily dosing or once daily dosing. Suitably, the Factor Xa inhibitor plasma level is exhibited for at least 24 hours after administration to enable once daily dosing.

The pharmaceutical compositions of the present invention comprise a plurality of mini-tablets (or “mini-tabs”), for example 2-30 mini-tablets, 4 to 22 mini-tablets, or 5 to 20 mini-tablets. Suitably, the mini-tablets in accordance with the invention are contained in a capsule or sachet for oral administration. Suitably, the capsule is a hard gelatin or hydroxymethylcellulose (HPMC) capsule. In one aspect of the invention, the capsule contains a particulate overfill, such as microcrystalline cellulose. In one aspect of the invention, 2 to 8 mini-tablets are provided within a capsule, for example 3 to 7 mini-tablets, 4 to 6 mini-tablets or 5 mini-tablets within a capsule. In another aspect of the invention, 7 to 14 mini-tablets are provided within a capsule, for example 8 to 13 mini-tablets, 9 to 12 mini-tablets or 10 mini-tablets in a capsule. An additional aspect of the invention includes 17 to 23 mini-tablets in a capsule, for example 18 to 22 mini-tablets, 19 to 21 mini-tablets or 20 mini-tablets in a capsule.

Suitably, the mini-tablets have a diameter of less than 5 mm, 4.5 mm or less, or less than 4.5 mm, for example 0.2 to 4.5 mm, 0.5 to 4.5 mm, 1 to 4.5 mm, 2 to 5 mm, 2 to 4.5 mm, 2 to 4 mm, 2 to 3.5 mm, 2.5 to 5 mm, 2.5 to 4.5 mm, 2.5 to 4 mm, 2.5 to 3.5 mm, 3 to 5 mm, 3 to 4.5 mm, 3 to 4 mm, 3 to 3.5 mm, 3.1 to 3.3 mm or 3.2 mm. Suitably, the mini-tablets have a thickness of 5 mm or less, 4.5 mm or less, or less than 4.5 mm, for example 0.2 to 4.5 mm, 0.5 to 4.5 mm, 1 to 4.5 mm, 2 to 5 mm, 2 to 4.5 mm, 2 to 4 mm, 2 to 3.5 mm, 2 to 3 mm, 2.4 to 2.6 mm or 2.5 mm. The mini-tablets may have any shape convenient to the skilled person e.g. spherical or cylindrical. In one aspect of the invention, the mini-tablets are round and convex (known in the art as “round standard convex”). For example, the mini-tablets may have the dimensions 3.2 diameter by 2.5 mm thick.

Pharmaceutical compositions of the present invention suitably comprise from 5 to 50% of a Factor Xa inhibitor, e.g. Compound A or Compound B, based on the total weight of the composition (unless otherwise stated, % compositions herein are based on the total weight of the core mini-tablet composition, including any film coating but excluding the capsule). In one aspect of the invention, the composition comprises from 10 to 45% of a Factor Xa inhibitor, e.g. Compound A or Compound B. In other aspects of the invention, compositions of the invention comprise from 15 to 40% of a Factor Xa inhibitor, from 20 to 40% of a Factor Xa inhibitor or from 30 to 40% of a Factor Xa inhibitor.

In one aspect of the invention, the total weight of the mini-tablet core is 20 mg and the total weight of the mini-tablet together with the enteric coating is 21.6 mg. A 20 mg mini-tablet may contain 5-10 mg of a Factor Xa inhibitor, for example 7.5 mg. A modified release composition comprising a plurality of mini-tablets may contain 25-175 mg, 30-40 mg, 60-90 mg or 125-175 mg of a Factor Xa inhibitor, e.g. Compound A or Compound B. For example, a modified release composition comprising a plurality of mini-tablets provided within a capsule may contain 37.5, 75, 150, 200, 250 or 300 mg of a Factor Xa inhibitor, e.g. Compound A or Compound B. Each mini-tablet may contain, for example 0.8-150 mg of the Factor Xa inhibitor.

The mini-tablet(s) of the present invention comprise a Factor Xa inhibitor within a matrix of polymer(s). The Factor Xa inhibitor is embedded or dispersed in the matrix polymer. Suitably the mini-tablets further comprise a filler, a lubricant, and a glidant (one or more such components may be utilized). In one embodiment, the present invention provides a pharmaceutical composition for oral administration comprising a plurality of mini-tablets, said mini-tablets having a diameter of 4.5 mm or less and comprising a therapeutically effective amount of a Factor Xa inhibitor homogenously integrated (or admixed) within a matrix comprised of one or more polymer(s).

Suitable matrix polymers include hydrophilic water soluble polymers, for example high molecular weight polymers (i.e. 100,000 to 800,000 daltons), such as hydroxypropyl methylcellulose polymers. HPMC is the abbreviation for hydroxypropyl methylcellulose, which has the official name of hypromellose in the USP and PhEur. Therefore, in one aspect on the invention, the matrix polymer is hydroxypropyl methylcellulose, such as Methocel™, for example Methocel™ K100M, Methocel™ K15M, or Methocel™ K4M, suitably Methocel™ K15M. Compositions of the invention suitably comprise from 20 to 60% matrix polymer. In one aspect of the invention, the composition comprises 20 to 50%, 20 to 40%, 25 to 40%, 20 to 30% or from 25 to 30% matrix polymer.

Suitably, the mini-tablet(s) further comprise a filler. Suitable fillers include microcrystalline cellulose. In one aspect of the invention, the filler is microcrystalline cellulose e.g. Avicel™ PH101. Avicel™ PH101 is microcrystalline cellulose with an average particle size of 50 μm. Compositions of the invention suitably comprise 20 to 50% filler. In one aspect of the invention, the composition comprises 20 to 40%, 25 to 40%, 20 to 30% or from 25 to 30% filler.

Suitably, the mini-tablet(s) further comprise a glidant. Suitable glidants include colloidal silicon dioxide and talc. In one aspect of the invention, the flow enhancer is colloidal silicon dioxide, for example Cab-O—Sil. Compositions of the invention suitably comprise from 0.1 to 5% glidant, based on the total weight of the composition. In one aspect of the invention, the composition comprises from 0.1 to 1% glidant.

Suitably, the mini-tablet(s) further comprise a lubricant. Suitable lubricants include stearic acid, and stearic acid salts, for example magnesium stearate. In one aspect of the invention, the lubricant is magnesium stearate. Compositions of the invention suitably comprise from 0.1 to 5% lubricant, based on the total weight of the composition. In one aspect of the invention, the composition comprises from 0.1 to 1% lubricant.

The mini-tablets may be uncoated, or coated with one or more layers of coating. Suitably, the mini-tablets are enteric coated. The enteric coating may comprise a pH dependent polymer, for example a copolymer of the methacrylic acid and methacrylic acid ester such as a methacrylic acid copolymer, for example Eudragit e.g. Eudragit L30D55 which has a dissolution above pH 5.5. Other Eudragits include: Eudragit L100-55 (dissolution above pH 5.5), Eudragit L100 (dissolution above pH 6.0) and Eudragit S100 (dissolution above pH 7.0). Suitably, the enteric coating comprises from 5 to 10% based on the total weight of the composition (dry polymer weight), suitably 6-8%. The enteric coating can be produced by spraying the enteric polymer on top of the above-described core mini-tablet.

Suitably, the enteric coating further comprises a plasticizer. Suitably, the pharmaceutical compositions of the present invention further comprise a plasticizer to aid in film formation during the film coating process, such as acetyl triethyl citrate or triethyl citrate, for example triethyl citrate (Citroflex). Compositions of the invention suitably comprise from 0.1 to 5% plasticizer, based on the total weight of the composition. In one aspect of the invention, the composition comprises from 0.1 to 1% plasticizer.

Suitably, the enteric coating further comprises a glidant. Suitably, the pharmaceutical compositions of the present invention further comprise a glidant to eliminate sticking during the film coating process such as talc, kaolin, or glycerol monostearate, for example glycerol monostearate (Imwitor 900K). Compositions of the invention suitably comprise from 0.1 to 5% glidant, based on the total weight of the composition. In one aspect of the invention, the composition comprises from 0.1 to 1% glidant.

Suitably, the enteric coating further comprises a surfactant. Suitably, the pharmaceutical compositions of the present invention further comprise of a surfactant to provide homogeneous film mixtures, such as sodium lauryl sulphate, polyethylene glycol, or polysorbate, for example Polysorbate 80 (Crillet 4HP). Compositions of the invention suitably comprise from 0.1 to 5% based on the total weight of the composition. In one aspect of the invention, the composition comprises from 0.1 to 1% surfactant.

The compositions of the invention may, if desired, further include one or more pharmaceutically acceptable excipients. All such excipients must be “pharmaceutically acceptable” in the sense of being compatible with the other ingredients of the pharmaceutical composition and not injurious to the patient. Pharmaceutically acceptable excipients may include colours, flavours e.g. menthol, sweeteners e.g. mannitol, preservatives, stabilisers, antioxidants and any other excipients known to those skilled in the art.

It is to be understood that the present invention covers all combinations of the above embodiments and aspects of the invention described herein above.

A further aspect of the invention provides a process for preparing a pharmaceutical composition according to the invention. The compositions of the invention are suitably prepared by, in one or more steps, combining the components, granulating, drying, milling, and compressing the mixture into tablets. In one embodiment, the compositions are prepared using a wet granulation method, such as are well known in the art. For example, the Factor Xa inhibitor, a filler, a polymer and sufficient amounts of a granulating fluid such as water are combined, granulated, dried and milled to form granules. The dried granules are milled to achieve a suitable particle size, for example a D50 (median particle size) between 50 to 300 microns (μm), for example 100-300 microns or 100-200 microns. The granules are then combined with the remaining components, for example using a high shear mixing process, and the mixture is compressed into the mini tablets. The tablets are then coated with an enteric coating composition and filled into capsules or directly filled into capsules without coating. The capsules may then be filled with a particulate overfill, such as microcrystalline cellulose.

The present invention also provides a pharmaceutical composition of the invention for the manufacture of a medicament for the treatment of a patient suffering from a condition susceptible to amelioration by a Factor Xa inhibitor.

The present invention also provides a pharmaceutical composition of the invention for use in the treatment of a condition susceptible to amelioration by a Factor Xa inhibitor

The present invention also provides a method of treating a patient suffering from a condition susceptible to amelioration by a Factor Xa inhibitor comprising administering a pharmaceutical composition of the invention.

In one aspect of the invention, the condition susceptible to amelioration by a Factor Xa inhibitor is selected from treatment of acute vascular diseases such as acute coronary syndromes including post-acute coronary syndrome (for example primary and secondary prevention of myocardial infarction and unstable angina and treatment of prothrombotic sequalae associated with myocardial infarction or heart failure), thromboembolism including venous thromboembolism (VTE) (deep vein thrombosis (DVT) and pulmonary embolism (PE)), acute vessel closure associated with thrombolytic therapy and percutaneous transluminal coronary angioplasty, transient ischemic attacks, peripheral arterial occlusion, prevention of vessel luminal narrowing (restenosis), and the prevention of thromboembolic events associated with atrial fibrillation, e.g. stroke (stroke prevention in patients with atrial fibrillation, SPAF).

In another aspect, the condition susceptible to amelioration by a Factor Xa inhibitor is selected from acute coronary syndromes (for example primary and secondary prevention of myocardial infarction and unstable angina and treatment of prothrombotic sequalae associated with myocardial infarction or heart failure), pulmonary embolism, deep vein thrombosis and the prevention of thromboembolic events associated with atrial fibrillation, e.g. stroke.

The term “treatment” and derivatives such as “treating” as used herein includes both treatment and prophylaxis.

For each of the above-indicated utilities and indications the amount required of a Factor Xa inhibitor will depend on a number of factors including the severity of the condition to be treated and the identity of the recipient and will ultimately be at the discretion of the attendant physician or veterinarian. Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. In general, however the composition is administered in an amount effective to treat or prevent conditions for which a Factor Xa inhibitor is indicated. In particular embodiments, from 30 mg to 1000 mg (especially 30 to 300 mg) of a Factor Xa inhibitor is administered daily.

In one embodiment, the composition is administered twice a day (e.g., every 8-16, 10-14, or 12 hours). For example, the above-mentioned daily doses are split for twice daily administration. In another embodiment, the pharmaceutical composition is administered once a day. In another embodiment, the pharmaceutical composition is administered in the fed state.

Factor Xa inhibitors may also be used in combination with other therapeutic agents. The invention thus provides, in a further aspect, a pharmaceutical composition comprising a Factor Xa inhibitor together with one or more further therapeutic agent(s). Factor Xa inhibitors may be used in combination with other antithrombotic drugs (such as thrombin inhibitors, thromboxane receptor antagonists, prostacyclin mimetics, phosphodiesterase inhibitors, fibrinogen antagonists, thrombolytic drugs such as tissue plasminogen activator and streptokinase, non-steroidal anti-inflammatory drugs such as aspirin, and the like), anti-hypertensive agents (such as angiotensin-converting enzyme inhibitors, angiotensin-II receptor antagonists, ACE/NEP inhibitors, β-blockers, calcium channel blockers, PDE inhibitors, aldosterone blockers), anti-atherosclerotic/dyslipidaemic agents (such as HMG-CoA reductase inhibitors) and anti-arrhythmic agents. In one aspect of the invention, the Factor Xa inhibitor is used in combination with a CYP3A4 inhibitor, such as ketoconazole, diltiazem or verapamil.

When a Factor Xa inhibitor is used in combination with a second therapeutic agent, the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. When combined in the same formulation it will be appreciated that the two compounds must be stable and compatible with each other and the other components of the formulation.

The present invention also provides a plurality of pharmaceutical compositions arranged in a pharmaceutical pack, conveniently with instructions for use.

In one embodiment, the composition is administered to a mammal, more particularly a human, in need thereof.

The present invention also extends to pharmaceutical compositions which are bioequivalent to the pharmaceutical compositions exemplified below, in terms of both rate and extent of absorption, for instance as defined by the US Food and Drug Administration and discussed in the so-called “Orange Book” (Approved Drug Products with Therapeutic Equivalence Evaluations, US Dept of Health and Human Services, 19th edn, 1999). A pharmaceutical composition which achieves an area under the curve (AUC) (90% confidence interval (Cl)) within the range 80-125% compared to the reference product is termed “bioequivalent”. The pharmaceutical composition may provide an in vivo “Area Under the Curve” (AUC) value which is equivalent to the pharmaceutical compositions exemplified below, for instance at least 80%, such as 80 to 125%, 90% to 125%, or 100% to 125%.

The following examples illustrate aspects of this invention but should not be construed as limiting the scope of the invention in any way.

### EXAMPLES

### Example 1

### Mini-Tablet Composition

The following table shows an enteric coated mini-tablet composition containing (E)-2-(5-chlorothien-2-yl)-N-{(3S)-1-[(1S)-1-methyl-2-morpholin-4-yl-2-oxoethyl]-2-oxopyrrolidin-3-yl}ethenesulfonamide (Compound A):

TABLE 1
Mini-tablet Composition

| | |
|---|---|
| Composition | mg/tablet |

Tablet Core

| | |
|---|---|
| Compound A | 7.50 |
| Hypromellose (Methocel K15M) | 6.00 |
| Microcrystalline Cellulose, Avicel PH101 | 6.24 |
| Colloidal Silica Dioxide (Cab-O-Sil) | 0.10 |
| Magnesium Stearate | 0.16 |

Enteric Coating

| | |
|---|---|
| Methacrylic Acid Copolymer Type C (Eudragit L30D55) | 1.37* |
| Triethyl Citrate (Citroflex 2) | 0.14 |
| Glycerol Monostearate (Imwitor 900K) | 0.06 |
| Polysorbate 80 (Crillet 4HP) | 0.03 |
| Total | 21.60 |

Each tablet contains 7.5 mg of Compound A. Various numbers of mini-tablets can be filled into capsules to deliver various capsule strengths. For example, for 150 mg strength, 20 mini-tablets in one capsule; for 75 mg strength, 10 mini-tablets; for 37.5 mg strength, 5 mini-tablets in one capsule. The capsule is a gelatin or hydroxymethylcellulose (HPMC) capsule.
*Dry polymer weight

Capsule Shell Gelatin, Red Iron Oxide (E172), Titanium Dioxide (E171).

### Process:

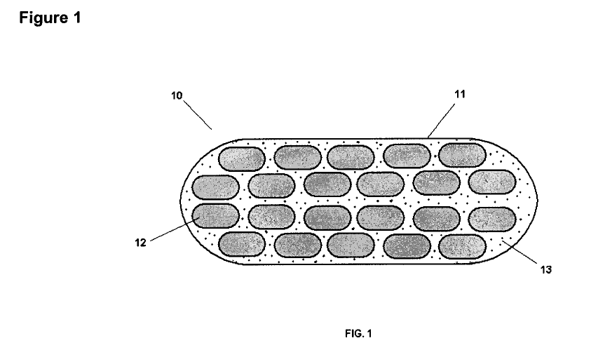

Drug was mixed with excipients and granulated using 45% (+15%) w/w of Purified Water. The dried granules were milled to achieve particle size D50 (median particle size) between 100-300 microns and blended with excipients and compressed into tablets. Enteric coating was carried out by mixing methacrylic acid copolymer with appropriate plasticizer, lubricant, and surfactant and coating by either wurster fluid bed coating or pan film coating. Mini-tablets were placed in capsules of either gelatin or hydroxymethylcellulose (HPMC) composition. FIG. 1 shows a diagram of an enteric coated mini-tablet pharmaceutical composition prepared according to the above process.

Uncoated mini-tablets were prepared as above without the enteric coating.

### Step by Step Procedure:

### Granulation:

1) Weighed out Drug Substance.
2) Weighed out Methocel, Avicel and screened them using a 20 mesh screen.
3) Transferred the ingredients to a high shear mixer-granulator.
4) Dried blend for 5-10 minutes (if necessary stopping in between to scrape off material from the container wall and then continuing to blend).
5) Checked bulk density of the dried blend: 0.248 g/ml.
6) Granulated with water until a suitable end point was reached. The spray rate target was 20-24 g/min/kg of material.
7) Wet-screen
8) Dried the granules until LOD of NMT 2.0% was reached.
9) Saved a sample (38 g) before milling to perform sieve analysis.
10) Milled the granules (screen size 024C, speed 1018 rpm, washer size 225).
11) Performed sieve analysis and bulk/tapped density testing on granules after milling (approx 96 g saved).

### Compression:

1) Weighed the granules, Cab-O—Sil and Magnesium stearate. Screened the Cab-O—Sil and Magnesium stearate using a 35-40 mesh screen.
2) Added the granules and Cab-O—Sil to a mixing container. Blended for 5-10 minutes at 25 rpm.
3) Added Magnesium Stearate to the mixing container. Blended for 5 minutes at 25 rpm.
4) Compressed tablets at an average compression force (KN) of 10 minutes at 1.0-1.1KN followed by 30 minutes at 0.9-1.0KN.

### Enteric Coating:

Dispensed the water into a suitable container (container 1). Heated the water to 70-80° C. Stirred the water using a suitable mixer. Slowly added Polysorbate 80, then Triethyl Citrate and then Imwitor 900K to the water vortex. Kept the mixture temperature 70-80° C. while stirring. Then allowed the mixture to cool to below 30° C. while continuing to slowly mix. Dispensed Eudragit L30D55 into a suitable container (container 2) and slowly stirred. Added the content in container 1 to container 2 under stirring and mixed for at least 30 minutes. Immediately prior to coating, the coating suspension was sieved through a 60 mesh screen. The cores were warmed at 25-35° C. and the suspension was continuously stirred during the coating process. The coating suspension was sprayed onto the cores to achieve the required specification, and coating stopped once sufficient film coat had been applied (application of the film coating suspension was controlled so that the exhaust temperature did not drop below 35° C.). The hot air supply to the inlet air was turned off and the tablets allowed to cool. Periodically the tablets were rotated in the pan whilst cooling.

### Example 2

### Pharmacokinetic (PK) Study

The PK properties of the pharmaceutical compositions according to Example 1 were evaluated in the following Pharmacokinetic Study.

### PK Methodology:

A 2-cohort, open-label, randomized, three-session, cross-over study in healthy subjects was performed. During each study session, subjects received a single oral dose of Factor Xa inhibitor (Compound A) as 150 mg strength dose administered in a fasted state, administered 30 min after the start of a light breakfast, or administered 30 min after start of a high fat breakfast. Each session was separated by a minimum washout period of 5-7 days. Samples for PK analysis were collected 0.25, 0.5, 0.75, 1, 1.5, 2, 3, 4, 6, 8, 10, 12, 18, and 24 hours post-dose. Plasma samples were assayed for Compound A using a validated HPLC-MS/MS assay method.

TABLE 2a
Composition of Light Breakfast (Standard Meal)

| | | Carbo- | | | |
|---|---|---|---|---|---|
| | | hydrate | | | |
| Food | Quantity | (g) | Protein (g) | Fat (g) | Calories |
| Cereal | 1 cup | 23 | 6 | 0 | 102 |
| (Special K) | | | | | |
| skimmed milk | 8 oz | 11.9 | 8.4 | 0.4 | 78 |
| Toast | 1 toast | 12 | 3 | 0.5 | 57 |
| low-fat spread | 1 tsp | 0 | 0 | 3.7 | 33 |
| fruit juice | ½ cup (4 oz) | 15 | 0 | 0 | 51 |
| (apple/orange) | | | | | |
| Total: | | 59.8 | 15.3 | 4.5 | 321 |

This meal is compliant with the FDA Guidance for Industry: Food-Effect Bioavailability and Fed Bioequivalence Studies

TABLE 2b
Composition of High Fat Breakfast (High Fat Meal)

| | | Carbo- | | | |
|---|---|---|---|---|---|
| | | hydrate | | | |
| Food | Quantity | (g) | Protein (g) | Fat (g) | Calories |
| 2 eggs fried in | 2 eggs/1 tsp | 1.2 | 12.6 | 10 + 7.6 | 213 |
| butter | butter | | | | |
| Bacon | 2 strips | 0 | 4 | 5 | 61 |
| hash brown | 4 oz | 15 | 3 | 1 | 80 |
| potatoes | | | | | |
| whole milk | 8 oz | 12 | 8 | 8 | 145 |
| Toast | 2 slices | 24 | 6 | 1 | 115 |
| pats of butter | 2 tsp | 0 | 0 | 7.6 | 68 |
| Total: | | 52.2 | 33.6 | 40.2 | 682 |

This meal is compliant with the FDA Guidance for Industry: Food-Effect Bioavailability and Fed Bioequivalence Studies

### Results

TABLE 3a
Summary of Pharmacokinetics for Enteric Coated Mini-tabs

| | | AUC(0-t) | Cmax | Tmax | C24 |
|---|---|---|---|---|---|
| Fasted | N¹ | 13 | 13 | 13 | 13 |
| | Median | 5149 | 428 | 12 | 104 |
| | Mean | 4858 | 418 | 8.9 | 125 |
| | CV % | 46% | 45% | 61% | 83% |
| Standard | N¹ | 13 | 13 | 13 | 12 |
| | Median | 5098 | 396 | 12 | 95 |
| | Mean | 4779 | 4278 | 8.5 | 88.4 |
| | CV % | 54% | 43% | 53% | 92 |
| High Fat | N¹ | 13 | 13 | 13 | 13 |
| | Median | 4832 | 505 | 12 | 168 |
| | Mean | 4938 | 454 | 9.4 | 131 |
| | CV % | 47% | 42% | 45% | 92% |

¹N refers to the number of patients receiving dose under each study period.

TABLE 3b
Enteric Coated mini-tablets two one-sided tests

| Parameter | †Treatment | GMR* | 90% Cl |
|---|---|---|---|
| AUCt | Std meal | 0.98 | (0.73-1.33) |
| | High Fat | 1.02 | (0.75-1.37) |
| Cmax | Std meal | 1.02 | (0.78-1.33) |
| | High Fat | 1.09 | (0.83-1.42) |

†Fasted as reference
*Geometric Mean Ratio

TABLE 4a
Summary of Pharmacokinetics for Un-coated Mini-tabs

| | | AUC(0-t) | Cmax | Tmax | C24 |
|---|---|---|---|---|---|
| Fasted | N1 | 15 | 15 | 15 | 14 |
| | Median | 5098 | 598.3 | 3 | 68.0 |
| | Mean | 5221 | 575.4 | 3.45 | 59.6 |
| | CV % | 31.2% | 31.0% | 29.3% | 96.8% |
| Standard | N1 | 15 | 15 | 15 | 13 |
| | Median | 5314 | 759.8 | 3 | 33.2 |
| | Mean | 5764 | 815.1 | 3.6 | 35.0 |
| | CV % | 31.7% | 29.9% | 24.9% | 54.3% |
| High Fat | N1 | 15 | 15 | 15 | 10 |
| | Median | 5982 | 987.3 | 8 | 24.9 |
| | Mean | 5697 | 921.8 | 7.3 | 31.2 |
| | CV % | 32.6% | 31.6% | 64.8% | 79.7% |

¹N refers to the number of patients receiving dose under each study period.

TABLE 4b
Uncoated mini-tablets two one-sided tests

| Parameter | †Treatment | GMR | 90% Cl |
|---|---|---|---|
| AUCt | Std meal | 1.10 | (0.92-1.33) |
| | High Fat | 1.09 | (0.91-1.32) |
| Cmax | Std meal | 1.42 | (1.18-1.70) |
| | High Fat | 1.60 | (1.33-1.92) |

†Fasted as reference

Conclusions:
Enteric coated mini-tablets show little effect of food, apart from a slight delay in the onset of absorption. This will be minimized upon repeat oral dosing.
“Zero-order”-like profile and demonstrates complete coverage over the dosing interval.

### Example 4

### Dissolution Testing

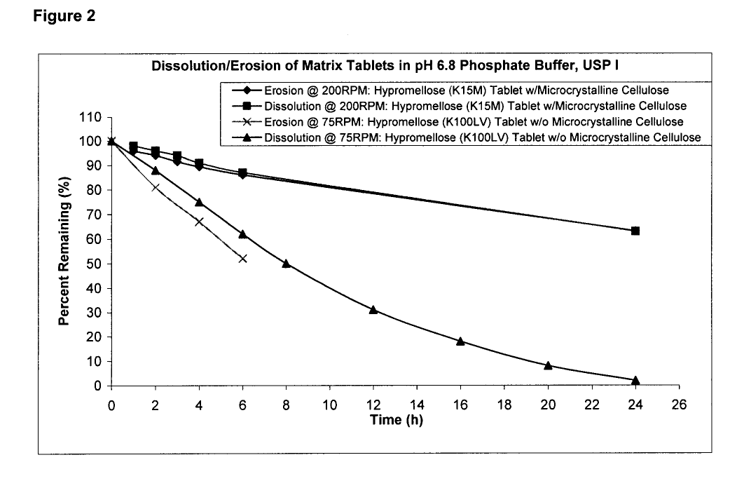

The dissolution profile according to FIG. 2 was generated using USP I Apparatus (Baskets) operating at 75 or 200 RPM speed, 37° C. temperature, and 900 ml phosphate buffer, pH 6.8.

The pharmaceutical composition containing K15M with (w) microcrystalline cellulose was run under more destructive conditions than the K100LV without (w/o) microcrystalline pharmaceutical composition (200 vs 75 rpm), and the K15M with microcrystalline cellulose pharmaceutical composition exhibited slower release and less erosion. This provides confidence that higher agitation rate in the stomach under fed conditions will be maintained with the pharmaceutical composition containing higher molecular weight polymer with microcrystalline cellulose.

TABLE 5
Dissolution Testing

| Dissolution @ 75 RPM | | | | | |
|---|---|---|---|---|---|
| (Hypromellose K100LV | | | Dissolution @ 200 RPM | | |
| without Microcrystalline | | | (Hypromellose K15M with | | |
| Cellulose) | | | Microcrystalline Cellulose) | | |
| Time | % | % | Time | % | % |
| (hr) | Remaining | Erosion | (hr) | Remaining | Erosion |
| 0 | 100 | 100 | 0 | 100 | 100 |
| 2 | 88 | 81 | 1 | 98 | 96 |
| 4 | 75 | 67 | 2 | 96 | 94 |
| 6 | 62 | 52 | 3 | 94 | 91 |
| 8 | 50 | | 4 | 91 | 89 |
| 12 | 31 | | 6 | 87 | 86 |
| 16 | 18 | | 24 | 63 | 63 |
| 20 | 8 | | | | |
| 24 | 2 | | | | |

All publications, including but not limited to patents and patent applications, cited in this specification are herein incorporated by reference as if each individual publication were specifically and individually indicated to be incorporated by reference herein as though fully set forth.

1. A modified release pharmaceutical composition for oral administration comprising a plurality of mini-tablets, said mini-tablets having a diameter of less than 5 mm and comprising a therapeutically effective amount of a Factor Xa inhibitor within a matrix of polymer(s).

2. A modified release pharmaceutical composition according to claim 1 wherein the mini-tablets have a diameter less than 4.5 mm.

3. A modified release pharmaceutical composition according to claim 1 wherein the mini-tablets are enteric coated.

4. A modified release pharmaceutical composition according to claim 1 wherein the matrix of polymer(s) are high molecular weight polymer(s) having a molecular weight from 100,000 to 800,000 daltons.

5. A modified release pharmaceutical composition according to claim 4 wherein the high molecular weight polymer is HPMC.

6. A modified release pharmaceutical composition according to claim 1 wherein the mini-tablets comprise 20 to 60% of matrix polymer(s) based on total weight of the composition excluding the capsule.

7. A modified release pharmaceutical composition according to claim 1 wherein the mini-tablets further comprise microcrystalline cellulose.

8. A modified release pharmaceutical composition for oral administration comprising a Factor Xa inhibitor having an in vivo maximum plasma concentration (Cₘₐₓ) following single oral dose administration to healthy adult humans wherein a ratio of Cₘₐₓ GMR Fasted:Fed is between 0.90 to 1.15.

9. A modified release pharmaceutical composition for oral administration comprising a Factor Xa inhibitor having an in vivo area under the curve (AUC) following single oral dose administration to healthy adult humans wherein a ratio of AUC GMR Fasted:Fed is between 0.90 to 1.15.

10. A modified release pharmaceutical composition for oral administration comprising a Factor Xa inhibitor and characterized by one or both of the following properties:
a) an in vivo maximum plasma concentration (Cₘₐₓ) following single oral dose administration to healthy adult humans wherein a ratio of Cₘₐₓ GMR Fasted:Fed is between 0.90 to 1.10; and
b) an in vivo area under the curve (AUC) following single oral dose administration to healthy adult humans wherein a ratio of AUC GMR Fasted:Fed is between 0.90 to 1.10.

11. A modified release pharmaceutical composition according to claim 1 wherein the Factor Xa inhibitor is (E)-2-(5-Chlorothien-2-yl)-N-{(3S)-1-[(1S)-1-methyl-2-morpholin-4-yl-2-oxoethyl]-2-oxopyrrolidin-3-yl}ethenesulfonamide and/or a pharmaceutically acceptable solvate thereof.

12. A modified release pharmaceutical composition according to claim 1 wherein each mini-tablet comprises 5 to 10 mg of a Factor Xa inhibitor.

13. (canceled)

14. (canceled)

15. A method of treating or preventing a condition susceptible to amelioration by a Factor Xa inhibitor comprising administering a modified release pharmaceutical composition according to claim 1.
